(19) United States
(12) Patent Application Publication
(10) Pub. No.: US 2020/0353452 A1
(43) Pub. Date: Nov. 12, 2020

(54) Catalyst and Method Related Thereto

(71) Applicant: SABIC Global Technologies B.V., Bergen op Zoom (NL)

(72) Inventors: Muhammad H. Haider, Riyadh (SA); Chandrasekar Subramani, Riyadh (SA); Khalid Karim, Riyadh (SA)

(21) Appl. No.: 16/764,438

(22) PCT Filed: Jan. 31, 2019
(86) PCT No.: PCT/IB2019/050796

§ 371 Date: May 15, 2020

Related U.S. Application Data
(60) Provisional application No. 62/626,441, filed on Feb. 5, 2018.

(51) Int. Cl. B01J 23/889; B01J 37/02; B01J 37/08; B01J 37/00; B01J 21/08; B01J 23/75; C07C 1/04

(52) U.S. Cl. CPC B01J 23/8892; B01J 37/0236; B01J 37/088; B01J 37/0072; B01J 37/0203; B01J 21/08; B01J 2523/72; C07C 1/0445; C07C 1/043; C07C 2521/08; C07C 2523/889; B01J 2523/845; B01J 23/75

(57) ABSTRACT

The present disclosures and inventions relate to a catalyst and method for producing and using the catalyst for the selective conversion of a hydrogen/carbon monoxide mixture (syngas) to C2+ hydrocarbons, while reducing the production of carbon dioxide.

### CROSS-REFERENCE TO RELATED APPLICATIONS

This application claims the benefit of U.S. Provisional Application No. 62/626,441, filed Feb. 5, 2018, which is incorporated herein by reference in its entirety.

### FIELD OF THE INVENTIONS

The compositions and methods disclosed herein relate to catalyst compositions and methods related thereto for the conversion of hydrogen/carbon monoxide mixtures (syngas) to hydrocarbons.

### BACKGROUND

Syngas (mixtures of H₂ and CO) can be readily produced from either coal or methane (natural gas) by methods well known in the art and widely commercially practiced around the world. A number of well-known industrial processes use syngas for producing various hydrocarbons and oxygenated organic chemicals.

The Fischer-Tropsch catalytic process for catalytically producing hydrocarbons from syngas was initially discovered and developed in the 1920's, and was used in South Africa for many years to produce gasoline range hydrocarbons as automotive fuels. The catalysts typically comprised iron or cobalt supported on alumina or titania, and promoters, such as rhenium, zirconium, manganese, and the like, were sometimes used with cobalt catalysts to improve various aspects of catalytic performance. The products were typically gasoline-range hydrocarbon liquids having six or more carbon atoms, along with heavier hydrocarbon products.

Today lower molecular weight hydrocarbons are desired and can be obtained from syngas via the Fischer-Tropsch catalytic process. Challenges exist to efficiently produce C2+ hydrocarbons at high yields without producing an excess of unwanted side products.

Accordingly, there remains a long-term market need for new and improved catalysts and methods related thereto for producing increased amounts of hydrocarbons, such as C2+ hydrocarbons, from syngas. Catalysts and methods useful for the production of hydrocarbons, such as C2+ hydrocarbons, from syngas are described herein.

### SUMMARY OF THE INVENTION

Disclosed herein is a method: a) mixing a suspension comprising a catalyst support and a solvent comprising water, with a cobalt salt or a manganese salt or a combination thereof, thereby forming a suspension comprising the catalyst support, cobalt or manganese or a combination thereof; and b) mixing the suspension comprising the support, cobalt or manganese or a combination thereof with urea, thereby producing a catalyst precursor.

Also disclosed herein is a catalyst prepared by the method disclosed herein.

Also disclosed herein is a composition comprising: a) a catalyst support; b) a solvent comprising water; c) a cobalt salt or a manganese salt or a combination thereof; and d) urea.

Additional advantages will be set forth in part in the description which follows, and in part will be obvious from the description, or can be learned by practice of the aspects described below. The advantages described below will be realized and attained by means of the chemical compositions, methods, and combinations thereof particularly pointed out in the appended claims. It is to be understood that both the foregoing general description and the following detailed description are exemplary and explanatory only and are not restrictive.

### DETAILED DESCRIPTION

Disclosed herein are materials, compounds, catalysts, compositions, and components that can be used for, can be used in conjunction with, can be used in preparation for, or are products of the disclosed method and compositions. It is to be understood that when combinations, subsets, interactions, groups, etc. of these materials are disclosed that while specific reference of each various individual and collective combinations and permutation of these compounds cannot be explicitly disclosed, each is specifically contemplated and described herein. For example, if a catalyst component is disclosed and discussed, and a number of alternative solid state forms of that component are discussed, each and every combination and permutation of the catalyst component and the solid state forms that are possible are specifically contemplated unless specifically indicated to the contrary. This concept applies to all aspects of this disclosure including, but not limited to, steps in methods of making and using the disclosed compositions. Thus, if there are a variety of additional steps that can be performed, it is understood that each of these additional steps can be performed with any specific aspect or combination of aspects of the disclosed methods, and that each such combination is specifically contemplated and should be considered disclosed.

In this specification and in the claims which follow, reference will be made to a number of terms which shall be defined to have the following meanings:

It must be noted that, as used in the specification and the appended claims, the singular forms “a,” “an” and “the” include plural referents unless the context clearly dictates otherwise. Thus, for example, reference to “a catalyst support” includes mixtures of catalyst supports.

“Optional” or “optionally” means that the subsequently described event or circumstance can or cannot occur, and that the description includes instances where the event or circumstance occurs and instances where it does not.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The word “or” as used herein means any one member of a particular list and also includes any combination of members of that list.

Ranges can be expressed herein as from one particular value, and/or to another particular value. When such a range is expressed, another aspect includes from the one particular value and/or to the other particular value. Similarly, when values are expressed as approximations, by use of the antecedent “,” it will be understood that the particular value forms another aspect. It will be further understood that the endpoints of each of the ranges are significant both in relation to the other endpoint, and independently of the other endpoint.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight of component Y, X and Y are present at a weight ratio of 2:5, and are present in such a ratio regardless of whether additional components are contained in the compound.

A weight percent of a component, unless specifically stated to the contrary, is based on the total weight of the formulation or composition in which the component is included.

The transitional phrase “consist essentially of” or “essentially consist of” limits the scope of the disclosure to the specified materials or steps and those that do not materially affect the basic and novel characteristic(s) of the invention.

1. Catalyst Precursor and Catalyst, and Method for Preparing Same

There is ongoing research to further develop sustainable technology of converting syngas to olefins, particularly light olefins, such as C2-C6 or C2-C4 olefins. Improving the catalyst used in this process is an important aspect of this development. Many catalytic regimes have been in focus over the past several years (E. Schwab, A. Weck, J. Steiner, K. Bay, Oil Gas Eur. Mag.1, 44-47 (2010); C. López, A. Corma, Chem. Cat. Chem. 4, 751-752 (2012); M. E. Dry, “The Fischer-Tropsch process: 1950-2000” Catalysis Today, vol. 71, pp. 227-241, January 2002). Cobalt based catalysts are of particular interest as they show efficient activity at low temperatures i.e. high conversion rates and long-term stability as compared to other catalyst regimes (F. Diehl, and A. Y. Khodakov, “Promotion of Cobalt Fischer-Tropsch Catalysts with Noble Metals: a Review,” Oil Gas Sci. Technol.-Rev. IFP vol. 64, no. 1, pp. 11-24, November 2008; Vannice, M. A. J. Catal. 1975, 37, 449). Different attempts have been made to further enhance and improve the efficiency and selectivity towards desired products to improve the cobalt based catalyst regime (James Aluha et al Industrial & Engineering Chemistry Research 2015 54 (43), 10661-10674; Gregory R. Johnson et al ACS Catalysis 2015 5 (10), 5888-5903).

The main reactions of a Fischer-Tropsch process can be carried out in a balanced manner (F. Fischer, H. Tropsch, Brennst.-Chem. 1923, 4, 276-285; F. Fischer, H. Tropsch, Brennst.-Chem. 1926, 7, 97-116; C. Knottenbelt, Catal. Today 2002, 71, 437-445). The Fischer-Tropsch process comprises of following main reactions:

2nH₂+nCO→CₙH₂ₙ+nH₂O (Alkenes) (1)

(2n+1)H₂+nCO→CₙH₂ₙ₊₂+nH₂O (Alkanes) (2)

Both of reactions 1 and 2 are highly exothermic and can be difficult to control. Apart from reactions 1 and 2, the water gas shift reaction (WGS) also has mechanistic importance in the Fischer-Tropsch process and influences the product selectivity of the heterogeneous catalysts used in the process (K.-W. Jun et al, Appl. Catalysis A: General, vol. 259, no. 2, pp. 221-226, 2004; N. Escalona, et al., Appl. Catalysis A: General, vol. 381, no. 1-2, pp. 253-260, 2010; M. Iglesias, et al., Catal. Today, vol. 215, pp. 194-200, 2013; B. H. Davis, Catal. Today, vol. 84, no. 1-2, pp. 83-98, 2003). The WGS is shown below in reaction 3.

CO+H₂O→CO₂+H₂ (WGS) (3)

The WGS reaction plays an important role in Fischer-Tropsch reactions for the production of olefins from syngas. This is twofold because the WGS acts as continuous source of hydrogen produced from water during the process. Other words, WGS is considered important for the efficient utilization of carbon monoxide in Fischer-Tropsch process (B. H. Davis, Catal. Today, vol. 84, no. 1-2, pp. 83-98, 2003; Borg et al., Appl. Catalysis B: Environmental, vol. 89, no. 1-2, pp. 167-182, 2009; E. de Smit and B. M. Weckhuysen, Chem. Soc. Rev., vol. 37, no. 12, pp. 2758-2781, 2008). However, the WGS also produce carbon dioxide, which is undesired because this considered waste and reduces the carbon efficiency of the process. As such, for industrial processes, it is desirable for the WGS to be balanced in order to attain desirable selectivity towards olefins at the same time minimizing the carbon waste in the form of carbon dioxide.

Disclosed herein is a method of producing a catalyst, such as a cobalt based catalyst, having a high olefin selectivity while simultaneously having a low selectivity towards the production of carbon dioxide. Such a catalyst is produced form a catalyst precursor.

Disclosed herein is a method of preparing a catalyst precursor. Also disclosed herein is a precursor catalyst prepared by the disclosed method. In one aspect, the catalyst precursor is a catalyst precursor suitable for use in a Fischer-Tropsch reaction. In one aspect, the catalyst precursor is a CoMn catalyst precursor. It is understood that the CoMn catalyst precursor is present in or on the catalyst support and that the catalyst support is also a part of the catalyst precursor.

Also, disclosed herein is a method of preparing a catalyst. Also disclosed, herein is a catalyst prepared by the disclosed method. In one aspect, the catalyst is a catalyst suitable for use in a Fischer-Tropsch reaction. In one aspect, the catalyst is a CoMn catalyst. It is understood that the CoMn catalyst is present in or on the catalyst support and that the catalyst support is also a part of the catalyst.

In one aspect, the CoMn catalyst precursor has the formula CoMnₓS_{y}O_{z}, wherein S is a catalyst support. It is understood that the catalyst support is a part of the catalyst precursor. In one aspect, the CoMn catalyst has the formula CoMnₓS_{y}O_{z}, wherein S is a catalyst support. It is understood that the catalyst support is a part of the catalyst.

The disclosed catalyst is for converting syngas to hydrocarbons, for example, selectively converting syngas to C2+ hydrocarbons, such as, for example, C₂-C₆ hydrocarbons or C₂-C₄ hydrocarbons. The catalyst disclosed herein has an improved conversion rate and selectivity for converting syngas to C2+ hydrocarbons, such as, for example, C₂-C₆ hydrocarbons or C₂-C₄ hydrocarbons, as compared to conventional catalysts. The catalyst disclosed herein also has a low selectivity for the production of CO₂, which is desired in a Fischer-Tropsch process, such as an industrial Fischer-Tropsch process.

In the composition comprising the CoMnₓS_{y}O_{z} catalyst, the molar ratio of manganese atoms to cobalt atoms, i.e. the value of “x” in the catalyst formula, can be from about 0.8 to about 1.2, from about 0.8 to about 1.1, from about 0.8 to about 1.0, from about 0.8 to about 0.9, from about 0.9 to about 1.2, from about 0.9 to about 1.1, from about 0.9 to about 1.0, from about 1.0 to about 1.2, or from about 1.0 to about 1.1. In one aspect, x can be about 1.0.

In the composition comprising the CoMnₓS_{y}O_{z} catalyst, the molar ratio of the catalyst support “S” atoms to cobalt atoms, i.e. the value of “y” in the catalyst formula, can be from about 0.01 to about 5.0, from about 0.1 to about 3.0, from about 0.1 to about 1.0, from about 0.3 to about 1.0, from about 0.5 to about 1.0, from about 0.7 to about 1.0, from about 0.1 to about 0.8, from about 0.3 to about 0.8, or from about 0.1 to about 0.5. In one aspect, y can be about 1.0 or about 0.5.

In one aspect, the molar ratio of x can be about 1.0 and the molar ratio of y can be from about 0.1 to about 1.0. In another aspect, the molar ratio of x can be from about 0.9 to about 1.1 and the molar ratio of y can be from about 0.1 to about 1.0. In yet another aspect, the molar ratio of x can be from about 0.9 to about 1.1 and the molar ratio of y can be from about 0.1 to about 0.8. In yet another aspect, the molar ratio of x can be from about 0.9 to about 1.1 and the molar ratio of y can be from about 0.5 to about 1.0.

In the composition comprising the CoMnₓS_{y}O_{z} catalyst, the molar ratio of oxygen atoms, i.e. the value of “z” in the catalyst formula, is a number determined by the valence requirements of Co, Mn, and catalyst support “S.” In one aspect, z is greater than 0 (zero). In another aspect, z can be 0 (zero). Even though a suitable catalyst composition of these inventions may be prepared or loaded into a reactor in the form of a mixed oxide (i.e. z is initially greater than 0), contact with hot syngas, either before or during the catalytic conversion of syngas to hydrocarbons begins, may result in the “in-situ” reduction of the catalyst composition and/or partial or complete removal of oxygen from the solid catalyst composition, with the result that z can be decreased to zero or zero. In one aspect, the value of z can be any whole integer or decimal fraction between 0 and 10. In some aspects of the catalyst described herein, z is greater than zero. In some aspects of the catalysts described herein, z can be from 1 to 5.

Also disclosed herein is a composition comprising the disclosed catalyst precursor and a catalyst support material. Also disclosed herein is a composition comprising the disclosed catalyst and a catalyst support material.

The composition comprising a catalyst having the formula CoMnₓS_{y}O_{z} disclosed herein have a low water gas shift activity as compared to conventional catalyst. The water gas shift reaction provides a source of H₂ and CO₂ at the expense of CO and H₂O. Thus, unwanted CO₂ is produced by the water gas shift reaction. The composition comprising a catalyst having the formula CoMnₓS_{y}O_{z} disclosed herein have a low water gas shift activity, thereby producing a low amount of CO₂ as shown herein. For example, the composition comprising a catalyst having the formula CoMnₓS_{y}O_{z} disclosed herein have a water gas shift reaction that produces less than 8% or less than 5% CO₂ or less than 4% CO₂ from the carbon monoxide feed. Accordingly, the composition comprising a catalyst having the formula CoMnₓS_{y}O_{z} disclosed herein can have a CO₂ selectivity that is less than 8% or less than 5% or less than 4%.

In one aspect, the composition consists essentially of a catalyst precursor or a catalyst having the formula CoMnₓS_{y}O_{z}, wherein the molar ratio of x is from about 0.8 to about 1.2; wherein the molar ratio of y is from about 0.01 to about 5.0; and wherein the molar ratio of z is a number determined by the valence requirements of Co, Mn, and the catalyst support “S”, and a catalyst support. For example, the composition can consist essentially of a catalyst precursor or a catalyst having the formula CoMnₓS_{y}O_{z}, wherein the molar ratio of x is from about 0.9 to about 1.1; wherein the molar ratio of y is from about 0.1 to about 1.0; and wherein the molar ratio of z is a number determined by the valence requirements of Co, Mn, and the catalyst support “S.”

The CoMnₓS_{y}O_{z} catalyst precursor catalyst and/or CoMnₓS_{y}O_{z} catalyst herein can be non-stoichiometric solids, i.e. single phase solid materials whose composition cannot be represented by simple ratios of well-defined simple integers, because those solids probably contain solid state point defects (such as vacancies or interstitial atoms or ions) that can cause variations in the overall stoichiometry of the composition. Such phenomena are well known to those of ordinary skill in the arts related to solid inorganic materials, especially for transition metal oxides. Accordingly, for convenience and the purposes of this disclosure, the composition of the potentially non-stoichiometric catalytically active solids described herein will be quoted in ratios of moles of the other atoms as compared to the moles of cobalt and manganese ions or atoms in the same composition, whatever the absolute concentration of cobalt and manganese present in the composition. Accordingly, for purposes of this disclosure, the value of “x” and “y” are molar ratios relative to each other, regardless of the absolute concentration of cobalt and manganese in the catalyst. Thus, the subscript numbers represents molar ratios.

In one aspect, the composition comprising the CoMnₓS_{y}O_{z} catalyst precursor or the CoMnₓS_{y}O_{z} catalyst, wherein S is a catalyst support, the catalyst support is typically catalytically inert, but typically provides physical support, strength and integrity to catalyst particles or pellets containing both the catalyst compositions and the catalyst supports, so that catalyst lifetimes and performances are improved. Suitable catalyst supports (“S”) for the CoMnₓS_{y}O_{z} catalyst precursor and CoMnₓS_{y}O_{z} catalyst comprises Al₂O₃, SiO₂, TiO₂, CeO₂, AlPO₄, ZrO₂, MgO, ThO₂, boehmite, silicon-carbide, Molybdenum-carbide, an alumino-silicate, kaolin, a zeolite, or a molecular sieve, or a mixture thereof. For example, S can comprise Al₂O₃, SiO₂, TiO₂, or ZrO₂. In another example, S can comprise SiO₂. In one aspect, the S does not comprise MgO.

In one aspect, the composition essentially consists of the CoMnₓS_{y}O_{z} catalyst precursor. In another aspect, the composition consists of the CoMnₓS_{y}O_{z} catalyst.

In one aspect, the composition essentially consists of the CoMnₓS_{y}O_{z} catalyst. In another aspect, the composition consists of the CoMnₓS_{y}O_{z} catalyst.

In one aspect, the catalyst precursor, such as the CoMnₓS_{y}O_{z} catalyst precursor, does not comprise Fe. In one aspect, the catalyst, such as the CoMnₓS_{y}O_{z} catalyst, does not comprise Fe.

Accordingly, disclosed herein is a method: a) mixing a first suspension comprising a catalyst support and a solvent comprising water, with a cobalt salt or a manganese salt or a combination thereof, thereby forming a second suspension comprising the catalyst support, cobalt or manganese or a combination thereof; and b) mixing the suspension comprising the support, cobalt or manganese or a combination thereof with urea, thereby producing a catalyst precursor.

Urea is used as a precipitating agent in the method, which produces the catalyst precursor and catalyst disclosed herein with desired selectivity of production of C2+ hydrocarbons, such as, for example, C₂-C₆ hydrocarbons or C₂-C₄ hydrocarbons, and CO₂.

In one aspect, the solvent consists essentially of water. In another aspect, the solvent consists of water. In one aspect, the solvent is essentially free of organic solvents. In one aspect, the solvent is essentially free of alcohols. For example, the solvent can be essentially free of butanol.

In one aspect, the method further comprises the steps of: c) drying the catalyst precursor; and d) calcining the catalyst precursor, thereby producing a catalyst.

Accordingly, also disclosed herein is a composition comprising a) a catalyst support; b) a solvent comprising water; c) a cobalt salt or a manganese salt or a combination thereof; and d) urea.

It is understood that the components in the disclosed composition can be further defined as described herein. For example, the catalyst support can comprise Al₂O₃, SiO₂, TiO₂, CeO₂, AlPO₄, ZrO₂, MgO, ThO₂, boehmite, silicon-carbide, Molybdenum-carbide, an alumino-silicate, kaolin, a zeolite, or a molecular sieve, or a mixture thereof, such as, for example, SiO₂; the solvent can consist essentially of water; and the first suspension can comprise a cobalt salt and a manganese salt.

In one aspect, the solvent has a temperature from about 20° C. to about 95° C. For example, the solvent can have a temperature from about 40° C. to about 95° C. In another example, the solvent can have a temperature from about 60° C. to about 95° C. In yet another example, the solvent can have a temperature from about 70° C. to about 95° C. In yet another example, the solvent can have a temperature from about 70° C. to about 90° C. In yet another example, the solvent can have a temperature from about 75° C. to about 85° C.

In one aspect, the second suspension comprises a cobalt salt. In another aspect, the second suspension comprises a manganese salt. In yet another aspect, the second suspension comprises a cobalt salt and a manganese salt.

The cobalt salt or manganese salt or combination thereof can be mixed with first suspension from a solution of cobalt salt or manganese salt or combination thereof. This concentration of each of the cobalt salt or manganese salt or combination thereof can be from about 0.01 M to about 5.0 M, for example, from about 0.5 M to about 2.0 M, or about 1.0 M.

In one aspect, the concentration of the catalyst support in the first suspension is at least about 0.005 g catalyst support per ml of solvent. For example, the concentration of the catalyst support in the first suspension can be at least about 0.01 g catalyst support per ml of solvent. In yet another example, the concentration of the catalyst support in the first suspension can be from about 0.005 g to about 0.05 g catalyst support per ml of solvent.

In one aspect, the mixing of the second suspension comprising the support, cobalt or manganese or a combination thereof with urea, thereby producing a catalyst precursor, comprises adding urea from a solution, such as an aqueous solution, to the second suspension. In one aspect, the urea solution is added drop-wise to the suspension. The solution of urea can comprise from about 25 wt % to about 75 wt % of urea, for example, from about 40 wt % to about 60 wt % of urea.

In one aspect, the second suspension has a pH from about 1.5 to about 3.5 before the mixing with the urea. In another aspect, the second suspension has a pH below 4.0. In one aspect, the second suspension has a pH from about 5.5 to about 7.5 after the mixing with the urea. The mixing of the second suspension and urea can be done for a prolonged period of time, for example from 10 hours to 30 hours.

In one aspect, the active metal composition comprises a cobalt and manganese. The active metal composition determines to composition of the catalyst precursor and catalyst. For example, when the active metal composition comprises a cobalt and manganese a CoMnₓS_{y}O_{z} catalyst precursor and CoMnₓS_{y}O_{z} catalyst can be obtained. Many suitable compounds comprising Co that are soluble in water can be suitable. Any cobalt (II) or (III) salt that is soluble in the solvent, can be used, and the use of cobalt (II) nitrate, cobalt tris(acetylacetonate), cobalt bis(acetylacetonate), cobalt (II) chloride, cobalt (II) bromide, cobalt (II) iodide, cobalt (II) acetate, cobalt (II) sulfate, and cobalt (II) diacetate or a combination thereof are a specific examples of a suitable Co compound that can be dissolved to provide a suitable solution comprising Co. Any manganese (II) or (III) salt that is soluble in the solvent can be used, and the use of manganese (II) nitrate or manganese (II) acetate are a specific examples of suitable Mn compounds that can be dissolved to provide a suitable solution comprising Mn.

In one aspect, the suspension comprises from about 0.1 mole % to about 2.0 mole %, such as for example, from about 0.5 mole % to about 1.5 mole %, of the cobalt salt prior to the formation of the catalyst precursor, such as the CoMnₓS_{y}O_{z} catalyst precursor. In another aspect, the solution comprises from about 0.1 mole % to about 2.0 mole %, such as for example, from about 0.5 mole % to about 1.5 mole %, of the manganese salt prior to the formation of the catalyst precursor, such as the CoMnₓS_{y}O_{z} catalyst precursor.

In one aspect, the method further comprises drying the catalyst precursor, such as the CoMnₓS_{y}O_{z} catalyst precursor. The drying of the catalyst precursor, such as the CoMnₓS_{y}O_{z} catalyst precursor can be done at a temperature from about 75° C. to about 175° C., such as, for example, from about 110° C. to about 150° C. In another aspect, the catalyst precursor, such as the CoMnₓS_{y}O_{z} catalyst precursor is filtered and washed prior to the drying step.

In one aspect of the methods for making the catalyst compositions, the method further comprises calcining the catalyst precursor, such as the CoMnₓS_{y}O_{z} catalyst precursor, thereby producing a catalyst, such as the CoMnₓS_{y}O_{z} catalyst. The calcining can be done in the presence of oxygen or air at high temperatures (such as for example exposing the catalyst composition to a temperature of from, about 200° C. to about 800° C.), or similar heating under a dry inert gas such as nitrogen, can also be required in order to fully form the catalyst compositions. For example, calcining can result in the conversion of a physical mixture of components to form the catalyst phase, via various chemical reactions, such as for example the introduction of oxygen atoms or ions into the composition. In one aspect, the method further comprises calcining the dried catalyst precursor, such as the CoMnₓS_{y}O_{z} catalyst precursor at a temperature from about 350° C. to about 650° C., to produce a catalyst, such as the CoMnₓS_{y}O_{z} catalyst.

As shown and described herein, the catalyst, such as a CoMnₓS_{y}O_{z} catalyst, resulting from the method disclosed surprisingly has improved properties, such as, improved conversion rate and selectivity for converting syngas to C₂-C₆ hydrocarbons, such as, for example, C₂-C₄ hydrocarbons, and a low selectivity for the production of CO₂, as compared to a catalyst, such as a CoMnₓS_{y}O_{z} catalyst, prepared using a conventional method.

It is also to be understood that in some aspects of the compositions and methods described herein, once a catalyst has been formed by the methods described above, and the formed catalyst is loaded into reactors and contacted with syngas at reaction temperatures for significant periods of time, some physical and chemical changes can occur in the catalyst, either quickly or over time as the catalytic reactions with syngas are carried out. For example, contact of the metal oxide catalysts described herein with syngas at high temperatures can cause partial or complete “in-situ” reduction of the metal oxides, and such reduction processes can cause removal of oxygen atoms from the solid catalyst lattices, and/or cause reduction of some or all of the metal cations present in the catalyst to lower oxidation states, including reduction to metallic oxidation states of zero, thereby producing finely divided and/or dispersed metals on the catalyst supports. Such reduced forms of the catalysts of the invention are within the scope of the described compositions and methods.

The possible components and ranges of components for such compositions have already been described above, and can be applied in connection with describing and claiming methods for preparing such compositions.

In view of the general descriptions of the preparations of the catalyst compositions and variations thereof that are part of these inventions described above, herein below are described certain more particularly described aspects of the inventions. These particularly recited aspects should not however be interpreted to have any limiting effect on any different claims containing different or more general teachings described herein, or that the “particular” aspects are somehow limited in some way other than the inherent meanings of the language and formulas literally used therein.

2. Methods for Producing Hydrocarbons from Syngas

Described above is a composition comprising a catalyst, for example a catalyst having the formula CoMnₓS_{y}O_{z} catalyst and methods for making such a catalyst. The catalyst is useful for converting mixtures of carbon monoxide and hydrogen (syngas) to hydrocarbons. The catalyst has unexpectedly high conversions of CO and selectivity for converting syngas to C2+ hydrocarbons, such as to low molecular weight hydrocarbons such as C₂-C₆ hydrocarbons, such as, C₂-C₄ hydrocarbons, and simultaneously have a low selectivity for the production of CO₂. In one aspect, the low molecular weight hydrocarbons such as C₂-C₆ hydrocarbons, such as, C₂-C₄ hydrocarbons are olefins.

Also disclosed herein is a method of producing C2+ hydrocarbons comprising contacting syngas with a composition comprising a catalyst having the formula CoMnₓS_{y}O_{z} catalyst, as disclosed herein, thereby producing C2+ hydrocarbons, such as C₂-C₆ hydrocarbons, such as, C₂-C₄ hydrocarbons.

In one aspect, the catalyst composition has a formula comprising a CoMnₓS_{y}O_{z} catalyst prior to introducing it to conditions suitable for contacting and reacting the catalyst composition with the syngas. Such conditions are known in the art and include high temperatures. The catalyst composition is reduced when present in the conditions associated with process of producing C2+ hydrocarbons by contacting the catalyst composition with syngas. Such catalyst composition is and can be referred to herein as a “reduced form of a catalyst composition comprising.” A reduction of the catalyst compositions under such conditions is known to those skilled in the art.

In these methods, mixtures of carbon monoxide and hydrogen (syngas) are contacted with suitable catalysts (whose composition, characteristics, and preparation have been already described above and in the Examples below) in suitable reactors and at suitable temperatures and pressures, for a contact time and/or at a suitable space velocity needed in order to convert at least some of the syngas to hydrocarbons. Unexpectedly as compared to methods in the prior art, the methods of the present inventions can be highly selective for the production of C2+ hydrocarbons, which are valuable feedstocks for subsequent cracking processes at refineries for producing downstream products, such as low molecular weight olefins. C2+ hydrocarbons can be C₂-C₁₂ hydrocarbons, C₂-C₈ hydrocarbons, C₂-C₆ hydrocarbons, C₂-C₄ hydrocarbons or C₂-C₃ hydrocarbons.

Methods for producing syngas from natural gas, coal, or waste streams or biomass, at almost any desired ratio of hydrogen to carbon monoxide are well known to those of ordinary skill in the art. A large range of ratios of hydrogen to carbon monoxide can be suitable for the practice of the current invention, but since high conversion of carbon monoxide to hydrocarbons is desired, syngas mixtures comprising at least equimolar ratios of hydrogen to carbon monoxide or higher are typically employed, i.e. from 3:1 Hz/CO to 1:1 Hz/CO. In some aspects, the ratios of hydrogen to carbon monoxide employed are from 2:1 Hz/CO to 1:1 Hz/CO. Optionally, inert or reactive carrier gases, such as N₂, CO₂, methane, ethane, propane, and the like can be contained in and/or mixed with the syngas.

The syngas is typically forced to flow through reactors comprising the solid catalysts, wherein the reactors are designed to retain the catalyst against the vapor phase flow of syngas, at temperatures sufficient to maintain most of the hydrocarbon products of the catalytic reactions in the vapor phase at the selected operating pressures. The catalyst particles can be packed into a fixed bed, or dispersed in a fluidized bed, or in other suitable arrangements known to those of ordinary skill in the art.

In one aspect, the syngas is contacted with the catalyst compositions at a temperature of at least 200° C., or at least 300° C., and at a temperature below 400° C. or from a temperature of 200° C. to 350° C., or from a temperature of 230° C. to 270° C.

In one aspect, the syngas is contacted with the catalyst compositions at a pressure of at least 3 bar, 5 bar, or at least, 10 bar, or at least 15 bar, or at least 25 bar, or at least 50 bar, or at least 75 bar, and less than 200 bar, or less than 100 bar. In many aspects of the methods of the reaction, the syngas is contacted with the catalyst compositions at a pressure from 5 bar to 100 bar. In many aspects of the methods of the reaction, the syngas is contacted with the catalyst compositions at a pressure from about 3 bar to about 15 bar.

In one aspect, the syngas is contacted with the catalyst compositions to produce relatively high conversions of the carbon monoxide present in syngas. In one aspect, conversion of carbon monoxide is at least 60%, at least 65%, at least 67%, at least 70%, at least 73%, or at least 75%. In one aspect, less than 8%, or less than 5% of the carbon monoxide fed to the reactors is converted to CO₂.

In one aspect, the methods disclosed herein are unexpectedly highly selective for the production of C2+ hydrocarbons. Typical C2+ hydrocarbons, detected in the product include saturated hydrocarbons such as methane, ethane, propanes, butanes, and pentanes, and unsaturated hydrocarbons such as ethylene, propylene, butenes, and pentenes. In another aspect, the methods disclosed herein are unexpectedly highly selective for the production of C2+ olefins, such as propylene. Typical C2+ olefins, detected in the product include ethylene, propylene, butenes, and pentenes. In one aspect, the method has an unexpectedly higher selectivity as compared to a reference catalyst not being prepared with a conventional solvent.

In one aspect, the selectivity for production of olefins can be from about 20% to about 45%, from about 32% to about 41%. In one aspect, the selectivity for production of C2-C4 olefins can be from at least about 10%, for example, from about 10% to about 25%, such as for example from about 15% to about 25%.

The production of methane in a Fischer-Tropsch process is undesired. In one aspect, the selectivity for production of CO₂ can be less than about 8%, less than about 5%, or less than about 4%.

In view of the general descriptions of the catalyst compositions and variations thereof that are part of the inventions described above, herein below are described certain more particularly described aspects of methods for employing the catalysts for converting syngas to hydrocarbons. These particularly recited aspects should not however be interpreted to have any limiting effect on any different claims containing different or more general teachings, or that the “particular” aspects are somehow limited in some way other than the inherent meanings of the language and formulas literally used therein.

3. Aspects

In view of the described catalyst and catalyst compositions and methods and variations thereof, herein below are described certain more particularly described aspects of the inventions. These particularly recited aspects should not however be interpreted to have any limiting effect on any different claims containing different or more general teachings described herein, or that the “particular” aspects are somehow limited in some way other than the inherent meanings of the language and formulas literally used therein.

Aspect 1: A method comprising the steps of: a) mixing a first suspension comprising a catalyst support and a solvent comprising water, with a cobalt salt or a manganese salt or a combination thereof, thereby forming a second suspension comprising the catalyst support, and cobalt or manganese or a combination thereof; and b) mixing the second suspension, thereby producing a catalyst precursor.

Aspect 2: The method of aspect 1, wherein the method further comprises the steps of: b) drying the catalyst precursor; and c) calcining the catalyst precursor, thereby producing a catalyst.

Aspect 3: The method of aspects 1 or 2, wherein the solvent has a temperature from about 20° C. to about 95° C.

Aspect 4: The method of aspects 1 or 2, wherein the solvent has a temperature from about 70° C. to about 95° C.

Aspect 5: The method of any one of aspects 1-4, wherein the catalyst support comprises Al₂O₃, SiO₂, TiO₂, CeO₂, AlPO₄, ZrO₂, MgO, ThO₂, boehmite, silicon-carbide, molybdenum-carbide, an alumino-silicate, kaolin, a zeolite, or a molecular sieve, or a mixture thereof.

Aspect 6: The method of aspect 5, wherein the catalyst support comprises SiO₂.

Aspect 7: The method of any one of aspects 1-6, wherein the second suspension.

Aspect 8: The method of any one of aspects 1-7, wherein the solvent is essentially free from organic solvents.

Aspect 9: The method of any one of aspects 1-8, wherein the concentration of the catalyst support in the first suspension is at least 0.005 g support per ml of solvent.

Aspect 10: The method of any one of aspects 1-9, wherein the mixing of the second suspension with urea comprises adding urea to the second suspension from an aqueous solution.

Aspect 11: The method of any one of aspects 1-10, wherein the first suspension has a pH below 4.0.

Aspect 12: The method of any one of aspects 2-11, wherein the step of drying is performed at a temperature from about 75° C. to about 175° C.

Aspect 13: The method of any one of aspects 2-12, where in the step of calcining is performed at a temperature from about 350° C. to about 650° C.

Aspect 14: A catalyst precursor produced by the method of any one of aspects 1 or 3-13.

Aspect 15: A catalyst produced by the method of any one of aspects 2-13.

Aspect 16: The catalyst of aspect 15, wherein the catalyst has the formula CoMnₓS_{y}O_{z}, wherein S is the catalyst support, wherein the molar ratio of x is from about 0.8 to about 1.2; wherein the molar ratio of y is from about 0.01 to about 5.0; and wherein the molar ratio of z is a number determined by the valence requirements of Co, Mn, and S.

Aspect 17: A method of producing C2+ hydrocarbons comprising contacting syngas with the catalyst of any one of aspects 15-16, thereby producing C2+ hydrocarbons.

Aspect 18: The method of aspect 17, wherein the method has a CO₂ selectivity of less than 8%.

Aspect 19: The method of aspect 17, wherein the method has a CO₂ selectivity of less than 5%.

Aspect 20: A composition comprising: a) a catalyst support; b) a solvent comprising water; c) a cobalt salt or a manganese salt or a combination thereof; and d) urea.

### EXAMPLES

The following examples are put forth so as to provide those of ordinary skill in the art with a complete disclosure and description of how the compositions, catalysts, and/or methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric.

The following lab grade chemicals were used without further purification; Fume silica (Aerosil 200V, Evonik Industries, Germany), Manganese (II) nitrate tetra hydrate (>97% purity, Sigma Aldrich), Cobalt (II) nitrate hexa hydrate (>98% purity, Sigma Aldrich), Urea (Technical grade, granular, Sabic), Magnesium chloride (1M soln.), Tetraethyl orthosilicate (TEOS), Ferric citrate, Ammonium hydroxide soln., H₂O₂ soln. (30 wt. %).

### 1. Example 1—Catalyst A

0.6 g of silica was suspended in 50 ml of demineralized water in a three necked round bottom flask and stirred for an hour at 90° C. 100 ml each of Co and Mn (1M) solutions were added to the above solution. The initial pH was 2.7. The suspension was then heated to 90° C. under vigorous stirring for 30 minutes. 300 ml (50 wt. %) of an aqueous solution of urea was added dropwise. The suspension was stirred for an additional 20 hours, before it was left to cool to room temperature. The pH was 6.4 at 90° C. The precipitate was filtered off, washed thoroughly with water and dried at 130° C. for 16 hours to give the catalyst precursor, which was subsequently calcined in static air at 500° C. (4 hours, 5° C./min), to produce the catalyst. This catalyst is denoted by the symbol “A” hereafter.

### 2. Example 2—Catalyst B

0.6 g of silica and 3.82 g of magnesium salt were suspended in 50 ml of demineralized water in a three necked round bottom flask and stirred for an hour at 90° C. 100 ml each of Co and Mn (1M) solutions were added to the above solution. The initial pH was 2.7. The suspension was heated to 90° C. under vigorous stirring. 300 ml (50 wt. %) of an aqueous solution of urea was added dropwise. The suspension was stirred for an additional 20 hours, before it was left to cool to room temperature. The pH was 6.4 at 90° C. The precipitate was filtered off, washed thoroughly with water and dried at 130° C. for 16 hours to give the catalyst precursor, which was subsequently calcined in static air at 500° C. (4 hours, 5° C./min), to produce the catalyst. This catalyst is denoted by the symbol “B” hereafter.

### 3. Example 3—Catalyst C

0.6 g of silica was suspended in 50 ml of demineralized water in a three necked round bottom flask and stirred for an hour at 90° C. 100 ml each of Co and Mn 1M solutions (prepared from the nitrate salts) were added to the above solution. The initial pH was 2.7. The suspension was heated to 90° C. under vigorous stirring. 300 ml (50 wt. %) of an aqueous solution of urea added dropwise. The suspension was stirred for an additional 20 hours, before it was left to cool to room temperature. The pH was 6.4 at 90° C. The precipitate was filtered off, washed thoroughly with water and dried at 130° C. for 16 hours to give material denoted as the catalyst precursor, which was subsequently calcined in static air at 400° C. (4 hours, 5° C./min). This catalyst is denoted by the symbol “C” hereafter.

### 4. Example 4—Catalyst D

0.6 g of silica was suspended in 50 ml of demineralized water in a three necked round bottom flask and stirred for an hour at 90° C. 100 ml each of Co and Mn 1M solutions (prepared from the nitrate salts) were added to the above solution. The initial pH was 2.7. The suspension was heated to 90° C. under vigorous stirring. 300 ml (50 wt. %) of an aqueous solution of urea added dropwise. The suspension was stirred for an additional 20 hours, before it was left to cool to room temperature. The pH was 6.4 at 90° C. The precipitate was filtered off, washed thoroughly with water and dried at 130° C. for 16 hours to give the catalyst precursor, which was subsequently calcined in static air at 500° C. (4 hours, 5° C./min), to produce the catalyst. The catalyst was washed with 5-10 wt. % H₂O₂ solution (immersed for 1 hour and filtered) and dried for 4 hour before testing. This catalyst is denoted by the symbol “D” hereafter.

### 5. Example 5—Catalyst E

0.6 g of silica and 2.13 g of ferrous citrate suspended in 50 ml of demineralized water in a three necked round bottom flask and stirred for an hour at 90° C. 100 ml each of Co and Mn 1M solutions (prepared from the nitrate salts) were added to the above solution. The initial pH was 3.5. The suspension was heated to 90° C. under vigorous stirring for 30 minutes. 500 ml (50 wt. %) of an aqueous solution of urea was added dropwise. The suspension was stirred for an additional 20 hours, before it was left to cool to room temperature. The pH was 7.5 at 90° C. The precipitate was filtered off, washed thoroughly with water and dried at 130° C. for 16 hours to give material denoted as the catalyst precursor, which was subsequently calcined in static air at 500° C. (4 hours, 5° C./min), to produce the catalyst. This catalyst is denoted by the symbol “E” hereafter.

### 6. Example 6—Catalyst F

Modified silica was prepared by taking 20 ml of 1 M magnesium chloride (diluted to 100 ml with dist. H₂O) in the beaker and stirring it vigorously at 50-60° C. Afterwards, TEOS (21 g) was dropped into the mixture. Ferric citrate crystals (0.25 g) were added and the mixture was allowed to stir for 2 hours. 5 M NH₄OH (100 ml) was added and the mixture was vigorously stirred for an additional 2 hours. The resulting solid material was filtered, washed with hot water, and dried overnight at 130° C. The dried material was immersed in 15% H₂O₂ (50 ml) for 1 hour followed by filtration and drying for 4 hours at 130° C. until ready to be used as support for catalyst preparations.

0.6 g of modified silica was suspended in 50 ml of demineralized water in a three necked round bottom flask and stirred for an hour at 90° C. 100 ml each of Co and Mn 1M solutions (prepared from the nitrate salts) were added to the above solution. The initial pH was 3.0. The suspension was heated to 90° C. under vigorous stirred for 30 minutes. 300 ml (50 wt. %) of an aqueous solution of urea was added dropwise. The suspension was stirred for an additional 20 hours, before it was left to cool to room temperature. The pH was 7.3 at 90° C. The precipitate was filtered off, washed thoroughly with water and dried at 130° C. for 16 hours to give the catalyst precursor, which was subsequently calcined in static air at 500° C. (4 hours, 5° C./min), to produce the catalyst. This catalyst is denoted by the symbol “F” hereafter.

### 7. Example 7—Catalyst G

0.6 g of silica was suspended in 50 ml of demineralized water and 25 ml of butanol in a three necked round bottom flask and stirred for an hour at 90° C. 100 ml each of Co and Mn 1M solutions (prepared from the nitrate salts) were added to the above solution. The initial pH was 3.0. The suspension was heated to 90° C. under vigorous stirring for 30 minutes. 300 ml of an aqueous solution of urea added dropwise. The suspension was stirred for an additional 20 hours, before it was left to cool to room temperature. The pH was 7.3 at 90° C. The precipitate was filtered off, washed thoroughly with water and dried at 130° C. for 16 hours to give the catalyst precursor, which was subsequently calcined in static air at 500° C. (4 hours, 5° C./min). The catalyst is denoted by the symbol “G” hereafter.

### 8. Results

Catalysts A-G were evaluated for their activity and selectivity along with short term, as well as long term studies of the catalyst stabilities. Prior to activity measurement, all of the catalysts were subjected to an activation procedure, which was performed at 350° C. with the ramp rate of 3° C. min⁻¹ for 16 h in 50:50 H₂/N₂ flow (WHSV: 3600 h⁻¹). Catalytic evaluation was carried out in a high throughput fixed bed flow reactor setup housed in a temperature controlled system fitted with regulators to maintain pressure during the reaction. The products of the reactions were analyzed via online gas chromatography analysis. The evaluation of catalysts A-G was carried out under the following conditions unless otherwise mentioned elsewhere; 240° C., 5 bar, WHSV: 1875 h⁻¹, H₂/CO ratio of 2. The mass balance of the reactions is calculated to be 95±5%.

The space velocity effect of the feed was also monitored during the activity and selectivity measurements of the catalysts A-G. The minimum reaction time duration of the catalytic evaluation was 200 hours, to determine the long-term stability of catalysts A-G, which is also an indication of the performance of catalysts A-G in industrial scale processes.

As described above, the precipitation of cobalt and manganese onto silica was done using urea as the precipitating agent. Also, different calcination temperatures were adopted, for example, 400° C. (catalyst A) and 500° C. (catalyst C). The results are shown in Table 1. As shown a surprisingly low amount of carbon dioxide being produced during the reaction. By increasing the space velocity, the conversion rate dropped down with an increase in the olefins selectivity from 27 to 41% while the carbon dioxide selectivity remains unchanged, see Table 1, catalyst A. The addition of a Fe dopant decreased the activity and olefins selectivity respectively, see Table 1, catalyst E. Fe is believed to be less active in Fischer-Tropsch reactions resulting in the 10% decrease in activity.

Also, using alcohol together with water did not improve the performance of the catalysts. Using a water/butanol mixture as the solvent increased the activity of the catalyst. However, the olefins selectivity dropped to almost half of the catalyst prepared with only water as the solvent. Also the paraffin selectivity was increased, see Table 1, catalyst G.

The effect of a MgO/silica support was evaluated. A MgO/silica catalyst support (Table 1, catalyst B) provided for a catalyst with an activity similar to the base catalyst, which utilizes a pure silica support (Table 1, catalysts A and C). A 5% increase in the olefins selectivity was observed, reaching 32%. However, the production of carbon dioxide increased 5 fold, as compared to the base catalyst (18%), at the expense of a decrease in paraffin selectivity. The inclusion of MgO adds redox sites on the catalyst surface, which are responsible for producing carbon dioxide under operating conditions. Using a MgO/silica catalyst support did not effect on overall results except a slight decrease in activity, see Table 1, catalyst F.

The performances of the evaluated catalysts are shown in Table 1.

TABLE 1

| Catalysts* | A | B | C | D | E | F | G |
|---|---|---|---|---|---|---|---|
| Conversion (mol. %) | 76/48¹ | 78/43² | 76 | 64/36¹ | 65 | 63 | 83 |
| | Selectivity (mol. %) | | | | | | |
| Olefins | 27/41 | 32/43 | 25 | 37/40 | 22 | 27 | 17 |
| Paraffins | 49/40 | 32/20 | 50 | 42/37 | 51 | 44 | 54 |
| Methane | 11/22 | 8/5 | 10 | 16/22 | 14 | 11 | 15 |
| C₂-C₄ | 10/17 | 10/7 | 12 | 15/17 | 15 | 12 | 11 |
| C₂═C₄ | 10/21 | 16/23 | 11 | 16/23 | 11 | 13 | 7 |
| C₅₊- | 39/34 | 21/13 | 39 | 39/28 | 36 | 32 | 42 |
| C₅₊ = | 16/22 | 16/20 | 14 | 19/18 | 11 | 15 | 10 |
| CO₂ | 4/3 | 18/21 | 6 | 4/2 | 4 | 6 | 4 |
| Olefins (% yield) | 20/19.5 | 25/18 | 18.5 | 23.7/15 | 14.2 | 17.1 | 14.2 |

*For details, see experimental section. Experiments were performed after activation under the following conditions: WHSV:1875 h⁻¹, H₂/CO:2, TOS 200 h. ¹WHSV:3000 h⁻¹, ²H₂:CO ratio:1.

The catalysts disclosed herein have a surprisingly low CO₂ selectivity, while olefins and paraffin remains major products. Thus, the catalysts disclosed herein solve the problem of CO₂ production in a syngas to olefins reaction, while the selectivity of the desired olefin and paraffin products remain.

Various modifications and variations can be made to the compounds, composites, kits, articles, devices, compositions, and methods described herein. Other aspects of the compounds, composites, kits, articles, devices, compositions, and methods described herein will be apparent from consideration of the specification and practice of the compounds, composites, kits, articles, devices, compositions, and methods disclosed herein. It is intended that the specification and examples be considered as exemplary.

What is claimed is:

1. A method comprising the steps of:
a) mixing a first suspension comprising a catalyst support and a solvent comprising water, with a cobalt salt or a manganese salt or a combination thereof, thereby forming a second suspension comprising the catalyst support, and cobalt or manganese or a combination thereof; and
b) mixing the second suspension, thereby producing a catalyst precursor.

2. The method of claim 1, wherein the method further comprises the steps of:
c) drying the catalyst precursor; and
d) calcining the catalyst precursor, thereby producing a catalyst.

3. The method of claim 1, wherein the solvent has a temperature from about 20° C. to about 95° C.

4. The method of claim 1, wherein the solvent has a temperature from about 70° C. to about 95° C.

5. The method of claim 1, wherein the catalyst support comprises Al₂O₃, SiO₂, TiO₂, CeO₂, AlPO₄, ZrO₂, MgO, ThO₂, boehmite, silicon-carbide, molybdenum-carbide, an alumino-silicate, kaolin, a zeolite, or a molecular sieve, or a mixture thereof.

6. The method of claim 5, wherein the catalyst support comprises SiO2.

7. The method of claim 1, wherein the second suspension.

8. The method of claim 1, wherein the solvent is essentially free from organic solvents.

9. The method of claim 1, wherein the concentration of the catalyst support in the first suspension is at least 0.005 g support per ml of solvent.

10. The method of claim 1, wherein the mixing of the second suspension with urea comprises adding urea to the second suspension from an aqueous solution.

11. The method of claim 1, wherein the first suspension has a pH below 4.0.

12. The method of claim 2, wherein the step of drying is performed at a temperature from about 75° C. to about 175° C.

13. The method of claim 2, where in the step of calcining is performed at a temperature from about 350° C. to about 650° C.

14. A catalyst precursor produced by the method of claim 1.

15. A catalyst produced by the method of claim 2.

16. The catalyst of claim 15, wherein the catalyst has the formula CoMnₓS_{y}O_{z}, wherein S is the catalyst support, wherein the molar ratio of x is from about 0.8 to about 1.2; wherein the molar ratio of y is from about 0.01 to about 5.0; and wherein the molar ratio of z is a number determined by the valence requirements of Co, Mn, and S.

17. A method of producing C2+ hydrocarbons comprising contacting syngas with the catalyst of claim 15, thereby producing C2+ hydrocarbons.

18. The method of claim 17, wherein the method has a CO₂ selectivity of less than 8%.

19. The method of claim 17, wherein the method has a CO₂ selectivity of less than 5%.

20. A composition comprising:
a) a catalyst support;
b) a solvent comprising water;
c) a cobalt salt or a manganese salt or a combination thereof; and
d) urea.
